(19) United States
(12) Patent Application Publication
(10) Pub. No.: US 2006/0258931 A1
(43) Pub. Date: Nov. 16, 2006

(54) Switching circuit arrangement comprising an integrated reference electrode and method for producing said switching circuit arrangement

(76) Inventor: Christian Paulus, Weilheim (DE)

Correspondence Address: DICKE, BILLIG & CZAJA, P.L.L.C., FIFTH STREET TOWERS, 100 SOUTH FIFTH STREET, SUITE 2250, MINNEAPOLIS, MN, 55402, US

(21) Appl. No.: 10/528,504

(22) PCT Filed: Sep. 19, 2003
(86) PCT No.: PCT/DE03/03123

§ 371 Date: May 16, 2006

(30) Foreign Application Priority Data
Sep. 19, 2002 (DE) 102 43 569.3

(51) Int. Cl. A61B 5/04; G01N 33/487

(52) U.S. Cl. 600/395; 600/396; 204/403.01

(57) ABSTRACT

The invention relates to a switching circuit arrangement and a method for producing a switching circuit arrangement. Said switching circuit arrangement contains a substrate comprising an integrated switching circuit, in addition to an integrated reference electrode which is formed on the substrate and has a metal core that is at least partially surrounded by an envelope consisting of a poorly soluble salt of the metal. The integrated switching circuit is electrically coupled to the core.

The invention relates to a circuit arrangement and a method for producing a circuit arrangement.

In chemical and biochemical analysis technology, electrochemical methods are often used in order to obtain qualitative or quantitative information about a concentration of a redox-active substance in an analyte. The reduction or oxidation potentials of the substances to be examined and also electric current values occurring at electrodes are often determined and evaluated in this case.

The electrochemical potential of a metallic electrode in a solution can be calculated using the Nernst equation, which describes the potential of a redox system in accordance with the following expression:
E=E₀+(RT)/(nF)log([Ox]/[Red]) (1)

In this case, E is the electrical potential of a redox system, E₀ is an electrical standard potential, R is the gas constant, T is the absolute temperature, n is the electrochemical valency, F is the Faraday constant, [Ox] is the concentration of the oxidized form and [Red] is the concentration of the reduced form of the redox substance.

It is evident from equation (1) that the electrochemical potential of a redox system depends on the concentration of the reactants. For this reason, it is necessary to provide a reference potential which forms a reference point for the electrochemical potentials independently of the concentration conditions in the analyte.

A reference potential of this type is provided by means of a reference electrode.

In the case of a reference electrode of a first type, the electrical potential is determined by the electrochemical equilibrium between the material of the electrode and its ions in the solution. The metal ions in the solution have a determining influence on the electrical potential. One example of a reference electrode of a first type is the hydrogen electrode. Clearly, in the case of an electrode of a first type, an electrically conductive structure enables the charge exchange between two media.

A known reference electrode of a second type is an arrangement in which the concentration of the ions that determine the electrical potential is defined by the presence of a sparingly soluble common-ion compound that supplies a second solid phase. A reference electrode of a second type is a metal which is covered with a layer of a sparingly soluble salt of the metal and is immersed in the solution of a readily soluble salt which has a common anion with the sparingly soluble salt. The electrode potential is defined by means of the activity of the anions of the readily soluble salt. Examples of reference electrodes of a second type are the calomel electrode or the silver/silver chloride electrode.

One embodiment of a silver/silver chloride electrode has a silver wire which is coated with silver chloride salt and is immersed in a solution containing chloride ions. Owing to the presence of the chloride ions in the solution, the concentration of the silver ions is very low and defined by the equilibrium
Ag⁺+Cl⁻ [symbol] AgCl (2).

In accordance with the prior art, macroscopic reference electrodes, in particular a silver/silver chloride electrode, which are brought into operative contact with the analyte are used in electrochemical analysis methods. Typical dimensions of electrodes of this type range from several centimeters to a few millimeters.

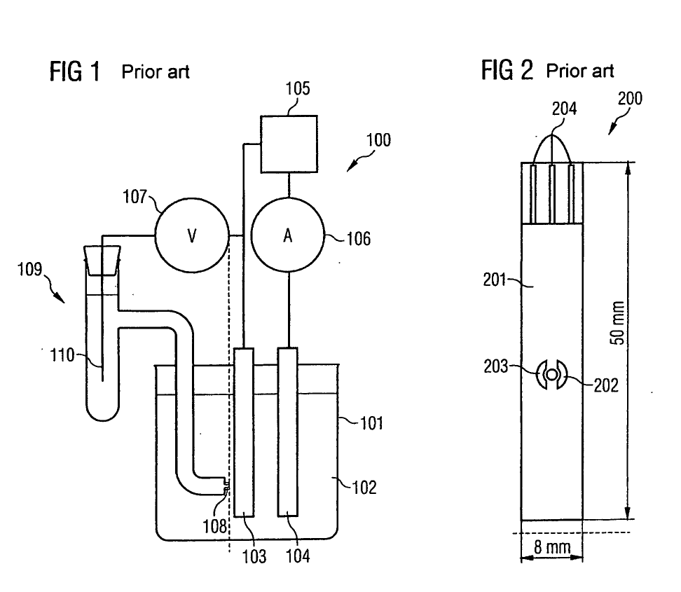

FIG. 1 shows an electrochemical experimental arrangement 100 in accordance with the prior art. An analyte 102 is filled into a container 101, which analyte is examined electrochemically by means of working electrodes 103, 104. The first working electrode 103 is coupled to a current supply 105, whereas the second working electrode 104 is coupled to an ammeter 106. Furthermore, a volt meter 107 is connected between the first working electrode 103 and a silver wire 110 of a silver/silver chloride electrode arrangement 109, said silver wire being coated with silver chloride material. The analyte 102 is brought into operative contact with the silver/silver chloride electrode arrangement 109 by means of a thin capillary 108. The analyte 102 of the electrochemical experimental arrangement 100 is provided with an electrical reference potential on account of the functionality of the silver/silver chloride electrode arrangement 109. Clearly, the experimental arrangement is set up for examining the analyte 102; a reference potential is provided for the examination by means of the silver/silver chloride electrode arrangement 109.

However, the silver/silver chloride reference electrode 109 shown in FIG. 1 has the disadvantage that a considerable minimum volume of the analyte 102 is required in order to reliably bring the macroscopic reference electrode into operative contact with the analyte 102. Furthermore, the mechanical construction of the silver/silver chloride electrode arrangement 109 is complicated, which is why reference electrodes of this type are only employed in laboratory systems. The silver/silver chloride electrode arrangement 109 is unsuitable for biosensor applications, in particular, since, as a rule, only small sample volumes are available in biology.

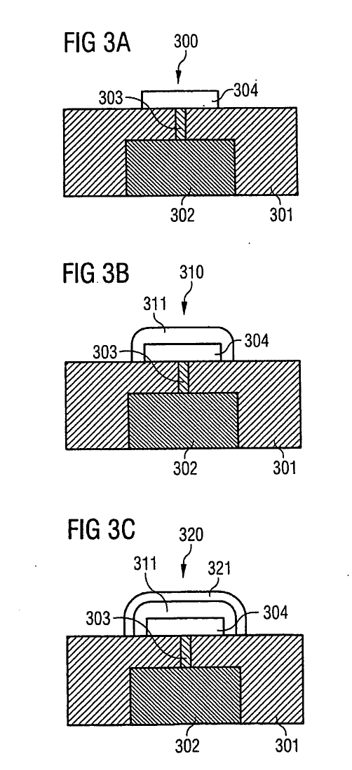

A description is given below, referring to FIG. 2, of a sensor arrangement 200 for electrochemical measurements which has a reference electrode, said sensor arrangement being disclosed in [1].

The sensor arrangement 200 has a dimensioning of 8 mm by 50 mm and has a working electrode 201, a counterelectrode 202 and a reference electrode 203. Contact-connections 204 are furthermore provided, by means of which the sensor arrangement 200 can be coupled to a control circuit. The sensor arrangement 200 has a three-electrode arrangement comprising the electrodes 201, 202, 203, which is customary in electrochemistry, and is produced using a screen printing method. The working electrode 201 is made of carbon, whereas the counterelectrode 202 and the reference electrode 203 are in each case formed as a silver/silver chloride electrode.

In order to determine the concentration of an analyte, the analyte is pipetted onto the electrodes 201 to 203, a volume of at least 15 μl being required. Sample volumes of this type are often not available at a sufficiently strong sample concentration, particularly in biological examinations.

In miniaturized systems, a chlorinated silver wire is often used as a reference electrode since such a wire can be formed with relatively small dimensions. In this case, a silver wire or a silver-coated platinum wire is superficially chlorinated and subsequently brought into operative contact with an analyte. As described in [2], for example, a reference electrode with dimensions in the millimeters range can thereby be formed, but an even higher degree of miniaturization is not possible. Therefore, the Dri-Ref™ reference electrodes disclosed in [2] are predominantly suitable for laboratory operation, whereas they are unsuitable for commercial applications.

Knowledge of the electrical potential of an electrolyte is important in particular in DNA sensor technology, for example in the case of redox recycling sensors. A reference electrode is therefore required in a DNA sensor arrangement if electrochemical analysis methods are used. In a DNA sensor, the diameter of the biologically activated region of a sensor arrangement is often between 100 μm and 200 μm, ten to a hundred DNA sensors typically being provided per mm² of substrate surface.

These dimensioning specifications result from the dimensioning that can be achieved when suitable DNA catcher molecules (e.g. DNA half strands) are applied to the sensor surface using a piezo-technology or needle technology. In the case of piezo-technology which is known illustratively as inkjet technology, the biological agent is applied to the sensor surface contactlessly in small droplets by means of a print head similar to those used in office inkjet printers. In the case of needle technology, which is referred to illustratively as stamp technology, the printing device operates in a manner comparable to a dot matrix printer. In a ring containing the biological agent to be printed, the printing needle is wetted with the liquid and subsequently pressed onto the sensor surface. Fundamental details about these technologies are described in [3], for example. [4] provides an overview of customary apparatuses and methods for producing microarrays.

[5] discloses a method for producing a combination of a pressure sensor and an electrical sensor.

[6] discloses an electrochemical sensor on a substrate with a electropositive metal electrode.

[7] discloses an electrochemical metal analysis device with a printed electrode.

[8] discloses a planar gas sensor, in particular a pCO₂ or pO₂ sensor.

[9] discloses a MOS transistor having a gate electrode that is electrically conductively coupled to an uncovered contact region.

The invention is based on the problem of providing a cost-effective reference electrode which can be produced with a tenable outlay and is also suitable for applications appertaining to biosensor technology.

The problem is solved by means of a circuit arrangement and by means of a method for producing a circuit arrangement having the features in accordance with the independent patent claims.

The circuit arrangement according to the invention has a substrate with a circuit integrated therein. Furthermore, the circuit arrangement of the invention has an integrated reference electrode—formed on the substrate—with a core that is made of a metal and is at least partly surrounded by a sheath made of a sparingly soluble salt of the metal. The integrated circuit is electrically coupled to the core.

Furthermore, the invention provides a method for producing a circuit arrangement, in which an integrated circuit is formed in a substrate, and in which an integrated reference electrode with a core made of a metal that is at least partly surrounded by a sheath made of a sparingly soluble salt of the metal is formed on the substrate. Furthermore, the integrated circuit is electrically coupled to the core.

Clearly, a miniaturized, monolithically integrated reference electrode of a second type is formed according to the invention, the integrated circuit of the circuit arrangement being electrically coupled to the reference electrode of a second type, with the result that the reference electrode can be brought to an electrical reference potential. The electrochemical reference electrode according to the invention is tailored in particular to the needs of large scale integrated electrochemical analysis systems. The reference electrode of the invention may advantageously be used on the surface of a circuit arrangement configured as a sensor arrangement, and may be formed in particular with dimensions comparable to those of integrated components (e.g. an integrated biosensor element). The outlay for producing the circuit arrangement according to the invention is low, with the result that the reference electrode is suitable in particular for mass production, e.g. of biosensor arrangements. The circuit arrangement makes it possible to provide a reference potential with a low outlay and with high accuracy. This is advantageous in particular with regard to the technology of an all electronic monolithic biosensor since no satisfactory solutions for providing a reference potential are known for such applications from the prior art. The miniaturized reference electrodes (which may be configured in particular as a silver/silver chloride reference electrode) can be produced with a low outlay (for example using simple printing or electrochemical methods). Complicated and therefore cost-intensive process technologies from the semiconductor industry are dispensable for the production of the electrode.

Preferred developments of the invention emerge from the dependent claims.

Preferably, the integrated circuit may be set up in such a way that a signal characteristic of the electrical potential in a region surrounding the reference electrode can be provided to said circuit by the reference electrode.

In other words, the reference electrode can detect the electrical potential in the region surrounding it (the surrounding region is e.g. an electrolyte in which the reference electrode is immersed) and provide a corresponding signal to the circuit. The circuit may furthermore be set up in such a way that it provides a control signal on the basis of the detected potential to a counterelectrode immersed in the electrolyte, for example, which control signal is chosen in such a way that the electrical potential of the electrolyte is influenced by means of the counterelectrode (e.g. the potential is set to the value of a reference potential).

The coupling between the integrated circuit and the core of the reference electrode may be realized as a direct coupling of an electrically conductive coupling means of the integrated circuit to the metallic core of the reference electrode.

As an alternative, the circuit arrangement may have an electrically conductive coupling structure by means of which the integrated circuit is electrically coupled to the metallic core. The coupling structure is preferably produced from a chemically inert material such as gold or platinum.

The metal of the core of the reference electrode is preferably silver, and the salt of the metal is preferably silver chloride. The reference electrode is then configured as a silver/silver chloride reference electrode. As an alternative, however, any other metal-metal salt combination is also suitable provided that the salt is sufficiently sparingly soluble (for example silver/silver iodide).

The substrate may have for example a semiconductor material (in particular silicon), glass, plastic and/or ceramic. The substrate is preferably a silicon wafer.

The circuit arrangement is preferably set up as a sensor arrangement. In such a case, the reference electrode of the circuit arrangement may serve for keeping an electrical potential of the sensor arrangement constant.

The circuit arrangement is further preferably set up as a biosensor arrangement. By way of example, the circuit arrangement may be a biosensor arrangement for DNA analysis using an electrochemical process (e.g. redox recycling). The reference potential required for the operation of the potentiostats contained on a semiconductor chip as substrate can be provided using the reference electrode according to the invention.

The method according to the invention for producing a circuit arrangement is described in more detail below. Refinements of the circuit arrangements also apply to methods for producing a circuit arrangement.

An electrically conductive coupling structure may be formed in such a way that the integrated circuit is electrically coupled to the core by means of said coupling structure.

Silver is preferably used as the metallic material of the core. Silver chloride is preferably used as the salt of the metal for forming the sheathing of the metallic core.

The core may be formed by means of printing silver material on the substrate and/or the coupling structure.

As an alternative, the core may be formed on the coupling structure using an electrodeposition method.

In accordance with a further alternative, the core may be formed by printing silver salt material on the substrate and/or the coupling structure using a printing technique known per se, and by reducing the silver salt material chemically to form silver.

Another alternative is for the core to be formed by forming a silver layer on the substrate and/or the coupling structure and patterning the silver layer (preferably using a lithography and an etching method).

In each of the alternatives described, the core can be at least partly surrounded by the sheath by chlorinating the core made of silver using an electrochemical method or a chemical method.

The method according to the invention makes it possible to integrate a reference electrode on and/or in the substrate. A subsequent, complicated fitting of the reference electrode is thereby dispensable. All materials on which electrically conductive structures can be applied as a component of the reference electrode are suitable as the substrate, such as glass, ceramic, plastic, for example. Use is preferably made of a semiconductor substrate which makes it possible to integrate the electrical circuit required for operating a sensor arrangement or for providing a constant voltage, preferably using CMOS technology.

Before the actual reference electrode is formed on the substrate, an electrically conductive coupling structure (also illustratively called basic electrode) is preferably applied on the chip surface. The basic electrode can be used to realize an electrical contact between the reference electrode to be formed and the circuit integrated in the substrate. The basic electrode is preferably produced from a chemically inert material such as gold or platinum.

In particular, in the case where the circuit arrangement is configured as a biosensor arrangement, it is advantageous to form the reference electrode of the invention in the same work operation and using the same or a similar device by means of which the surface of the substrate is biologically activated. In particular, a printable reference electrode is advantageous.

Exemplary embodiments of the invention are illustrated in the figures and are explained in more detail below.

In the figures:

FIG. 1 shows an electrochemical experimental arrangement in accordance with the prior art,

FIG. 2 shows a sensor arrangement in accordance with the prior art,

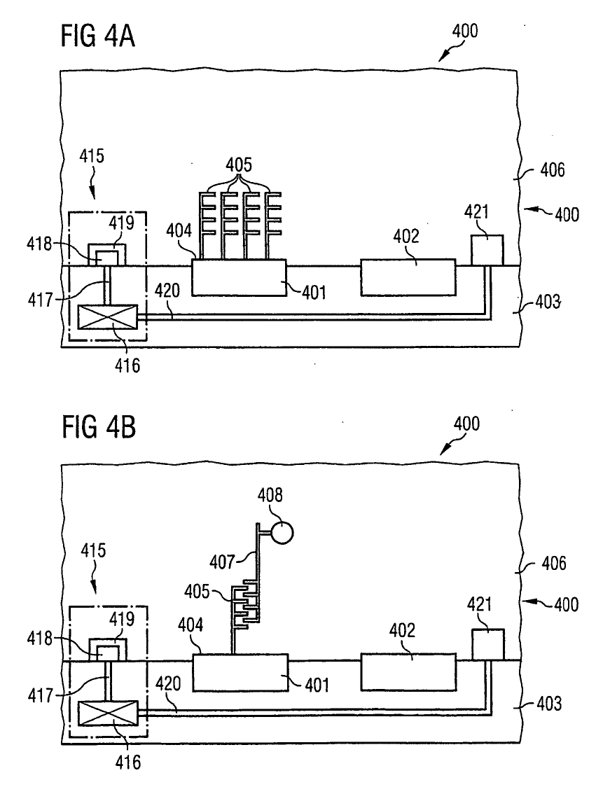

FIGS. 3A to 3C show layer sequences at different points in time during a method for producing a circuit arrangement in accordance with a preferred exemplary embodiment of the invention,

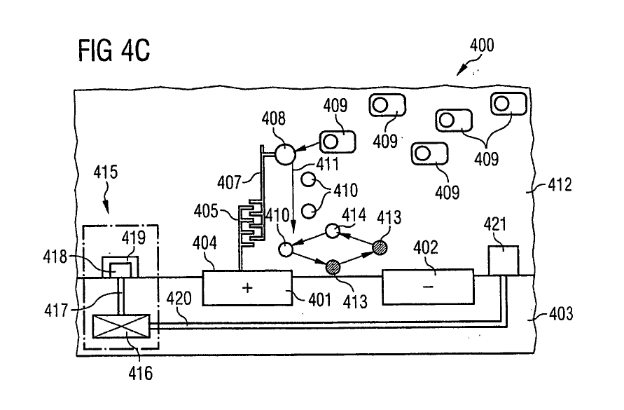

FIGS. 4A to 4C show a circuit arrangement configured as a redox recycling biosensor arrangement in accordance with a preferred exemplary embodiment of the invention.

In order to produce a circuit arrangement according to the invention, having a reference electrode made of a silver core and a silver chloride sheathing, firstly the core made of silver is formed on the substrate. The silver core is then at least partly surrounded by a sheath made of sparingly soluble silver chloride.

Firstly, a description is given of exemplary embodiments with regard to how metallic silver can be deposited onto a substrate in order to form a silver core on a substrate.

The silver core can be formed by means of printing silver material on the substrate (or by means of printing silver material on a previously formed coupling structure on the substrate for producing an electrical coupling between the integrated circuit of the substrate and the silver core).

For this purpose, by way of example, a solution of silver particles in a nitrocellulose lacquer is printed onto the substrate or onto the coupling structure (basic electrode), in which case it is possible to use the stamp method described above or the inkjet method described above. After a drying process an electrically conductive coating is formed on the substrate or on the basic electrode. This coating may serve directly as the metal core of the reference electrode or, as an alternative, be additionally reinforced galvanically. For galvanic reinforcement, the layer sequence obtained is immersed in a solution of a silver salt, and an electrical voltage is applied to the metallic core. Said voltage may be applied for example using the functionality of the integrated circuit already formed in the substrate. In accordance with this procedure, the form of the reference electrode formed can be set in a flexible manner by means of adjusting the printing process. This method has the advantage that the same printing technology that is also used for the biological activation of a sensor array can be used for the application of the silver core. Preferably, the silver core of the reference electrode is applied in the same work step in which the sensor arrays of a circuit arrangement configured as a biosensor arrangement are also provided with suitable capture molecules (for example DNA half strands). A special print head cleaning step may optionally be necessary if the application of silver material to the substrate or to the basic electrode is realized using an organic solution of silver particles, whereas an aqueous solution of the biomolecules is often used in the case of biological agents.

As an alternative, it is possible to form the silver core on the basic electrode (coupling structure) using an electrodeposition method.

In the case of this electrochemical method, elemental silver is deposited on the basic electrode (which is preferably made of gold material). For this purpose, the basic electrode is immersed in a silver salt solution (for example potassium cyanoargentate, K[Ag(CN₂]). After application of a suitable electrical potential with respect to a counterelectrode likewise immersed in the salt solution, the silver ions are chemically reduced at the basic electrode, with the result that elemental silver material is deposited on the basic electrode as core of the reference electrode. The geometrical form of the silver core corresponds to the form of the basic electrode in this case. By means of setting the duration of the electrodeposition or by means of setting the electric current value, it is possible to adjust the thickness of the resulting silver layer (core) on the basic electrode.

The electroplating of the basic electrode is preferably effected, before individual chips are sawn from the wafer substrate, in a scenario in which a plurality of circuit arrangements according to the invention are formed on a common substrate. Preferably, all (or some) of the basic electrodes are temporarily electrically coupled to one another using suitable electrical coupling means between the individual chips of the wafer substrate. Different integrated circuits coupled to different basic electrodes can also be electrically coupled to one another (e.g. by means of cross-chip interconnects). By means of immersing the wafer substrate in the silver salt solution and by means of applying a suitable voltage to the electrically coupled basic electrodes, silver is deposited on all the basic electrodes of all the chips on the wafer substrate in a common method step. In the course of subsequently sawing out the chips, it is possible to sever the electrically conductive coupling means between the basic electrodes (e.g. cross-chip interconnects). An alternative procedure for coating the basic electrodes with silver by electroplating may be performed for example with the use of interdigital electrodes (e.g. structures intermeshing in meandering fashion). If a plurality of electrodes are already present in sufficient proximity to one another on the chip surface, then a separate counterelectrode for electroplating is dispensable. In this case, it suffices to apply the negative electrical potential to the electrode to be silver-coated and to use at least one of the other electrodes, which are likewise in contact with the silver salt solution, as counterelectrode. In this case, the electrodes can be wetted with a silver salt solution after the sawing-out of the chip. If a suitable electrical voltage is applied, then silver is deposited on the basic electrode.

In accordance with a further alternative method for forming a silver core on the substrate (or on the basic electrode), the silver core is formed by printing silver salt material on the substrate and/or the basic electrode and chemically reducing the silver salt material to form silver. This method may illustratively be referred to as deposition of a silver mirror. For this purpose, a silver salt solution, for example an ammoniacal silver nitrate solution, is printed onto the basic electrode, and a reducing agent, for example a hydrazine solution (H₂N—NH₂) is added directly afterward. As a result, the silver ions contained in the salt solution are chemically reduced, with the consequence that elemental silver is deposited as the core of the reference electrode according to the invention on the chip surface wetted by the salt solution. This method has the significant advantage that electroplating is dispensable for depositing a closed silver layer on the basic electrode. In this case, the quality of the silver layer depends to a great extent on the chemical conditions under which the reduction reaction proceeds, and also on the constitution of the surface on which the silver mirror is deposited. The geometrical form of a reference electrode formed in this way is determined by the geometrical form of the printed-on silver salt solution and may be formed independently of the form of the basic electrode.

The silver salt solution, on the one hand, and the reducing agent, on the other hand, may also be printed on in already mixed form. If an ammoniacal silver nitrate solution (AgNO₃ with NH₃) is mixed with glucose solution and this mixture is printed onto the basic electrode, then a silver mirror is deposited on the printed surface. This reaction is substantially accelerated by heating the mixture after printing it on or printing it onto an already heated surface. If silver salt solution and reducing agent are printed on successively, then the deposition of the silver mirror can be considerably accelerated by means of suitable substance selection, in particular of the reducing agent, and by means of setting the concentrations, with the result that heating the mixture for silver-coating is also dispensable.

This method also has the major advantage that the same printing technology that is used for the biological activation of the sensor arrays (e.g. application of DNA half strands) can be used for the application of the silver electrode. Ideally, the silver electrode may even be applied in the same work step as the biological molecules. It is furthermore advantageous that exclusively an aqueous solution of the substances used is involved, with the result that no particular measures are required for cleaning the print head, as is the case for example when using organic solvents.

In accordance with a further alternative method for forming a metallic core on the substrate (or on the basic electrode), a silver layer is formed on the substrate and/or the coupling structure and the silver layer is patterned in such a way that a silver core remains.

In particular, a closed silver layer can be deposited on the surface of the substrate or on the basic electrode. A lithography and an etching method can be used to predefine a desired geometrical arrangement of the silver core or the silver cores on the surface of the substrate. Silver material that is free of a covering with photoresist is removed by means of the etching method, whereas silver material that is covered with photoresist is protected from etching.

A description is given below of exemplary embodiments of methods with regard to how the silver core formed in accordance with one or other of the methods described above is at least partly surrounded by a silver chloride sheath.

This may be realized by chlorinating the silver core using an electrochemical method.

In the case of electrochemical chlorination, the silver core is immersed in a solution containing chloride ions, for example sodium chloride (NaCl) or calcium chloride (KCl). Since silver is a relatively electropositive metal, an electrical voltage is preferably to be applied to the electrodes, so that the reaction
Ag+Cl⁻→AgCl+e⁻ (3)
proceeds sufficiently rapidly. The sodium material (Na⁺+e⁻→Na) or potassium material (K⁺+e⁻→K) arising at the counterelectrode reacts in the aqueous solution to form sodium hydroxide solution (NaOH) or potassium hydroxide solution (KOH), hydrogen being liberated:
2Na+2H₂O→2NaOH+H₂ (4)
or
2K+2H₂O→2KOH+H₂ (5)

As already described for the case of silver electroplating of the basic electrode, the silver core is preferably chlorinated before the chips are sawn from a wafer. Firstly all the basic electrodes (or a portion of the basic electrodes) are electrically coupled to one another by means of suitable electrically conductive coupling means between the chips on the wafer. All the silver electrodes of all the chips of the wafer are chlorinated in a common method step by means of immersing the wafer in a suitable salt solution containing chloride ions and by means of applying a suitable voltage to the electrode arrangement thus formed. In the course of subsequently sawing out the chips, the electrical couplings between the basic electrodes are automatically severed. The galvanic chlorination of the silver electrode can also be realized on the individual chip if a plurality of electrodes are present in sufficient proximity to one another on the chip electrode. A chloride-containing salt solution (for example NaCl or KCl solution) is pipetted onto the silver cores, a positive electrical potential is applied to the silver cores (preferably using the integrated circuits coupled thereto), and one of the other electrodes, which is likewise in operative contact with the salt solution is used as the counterelectrode.

As an alternative, the silver core may be at least partly surrounded by the sheath by chlorinating the silver core using a chemical method.

The chemical chlorination may be realized by immersing the silver core in, for example, an acidic iron(III) chloride solution, thereby initiating a chlorination reaction of the silver. The redox system Ag/Ag+ has a standard potential of 0.799V, and the redox system Fe³⁺/Fe²⁺ has a standard potential of 0.711V (pH=1), with the result that an independent chlorination proceeds only when the silver potential is reduced for example by coordination with complex ligands. Thus, the redox system Ag/AgCl has a standard potential of only 0.222V. This means that the silver core can be chlorinated by means of simply printing out an acidic iron chloride solution (FeCl₃). The redox equations for this reaction read as follows:
Ag−Ag⁺+e⁻ (E₀=0.222V in the presence of Cl⁻) (6)
Fe³⁺+e⁻→Fe²⁺ (E₀=0.771 volts at pH=1) (7)

This method also has the advantage that preferably the same printing technology that is used for the biological activation of sensor arrays is utilized for the chlorination of the silver core. The iron(III) chloride solution can be applied in the same work step as the biologically active capture molecules. It is furthermore advantageous that exclusively aqueous solutions of the substances used are used. As a result, it is possible to clean the print head without particular measures, as is the case for example when using organic solvents.

A further chlorination method that shall be mentioned, furthermore, is the direct chlorination of the silver electrode, in the case of which use is made of substances containing elemental chlorine, such as aqua regia (HNO₃+3HCl), for example. On account of the high reactivity of such substances, particular measures are necessary in particular during printing.

A description is given below, referring to FIG. 3A to FIG. 3C, of a preferred exemplary embodiment of the method according to the invention for producing a circuit arrangement.

In order to obtain the layer sequence 300 shown in FIG. 3A, firstly an integrated circuit 302 is formed in a silicon substrate 301. Using a suitable etching method, a passage hole is etched into the surface of the silicon substrate 301 and this contact hole is filled with a tungsten contact-connection element 303. The surface of the layer sequence thus obtained is then covered with a continuous gold layer and the gold layer is etched back using a lithography and an etching method in such a way that the gold basic electrode 304 remains as a result. As shown in FIG. 3A, the gold basic electrode 304 is coupled to the integrated circuit 302 via the electrically conductive tungsten contact-connection element 303.

In order to obtain the layer sequence 310 shown in FIG. 3B, silver material 311 is deposited as a core of a reference electrode on the gold basic electrode 304. In accordance with the exemplary embodiment described, this is realized by printing a silver salt solution on the gold basic electrode 304. Afterward, the silver salt material is chemically reduced to form elemental silver material using a reducing agent, as a result of which clearly a silver mirror 311 is formed. In accordance with the exemplary embodiment described, an ammoniacal silver nitrate solution (AgNO₃ with NH₃) is used as the silver salt solution and a glucose solution is used as the reducing agent.

In order to obtain the circuit arrangement 320 shown in FIG. 3C, the core made of silver material 311 is chlorinated using a chemical method, thereby forming the silver chloride sheathing 321. The chlorination is effected by means of adding iron(III) chloride, thereby forming a thin, porous silver chloride layer 321. The silver core 311 and the silver chloride sheathing 321 form an integrated reference electrode, to which an electrical coupling with the integrated circuit 302 is realized by means of the electrically conductive basic electrode 304 and by means of the electrically conductive tungsten contact-connection element 303. The integrated circuit 302 is set up in such a way that a signal characteristic of the electrical potential in a region surrounding the reference electrode can be provided to said circuit by the reference electrode.

The method described is distinguished by a particularly simple procedure: all the substances are present in aqueous solution, for which reason they are suitable for the printing methods that are also customary in biotechnology. In particular, complicated electro-chemical processes (electroplating, chlorination) are dispensable, for which reason the production method described is very cost-effective. The silver mirror 311 has a sufficiently good mechnical stability. In particular, the circuit arrangement 320 is suitable for being operated jointly with a biosensor that may preferably also be integrated on or in the substrate 301.

A description is given below, referring to FIG. 4A to FIG. 4C, of a circuit arrangement configured as a biosensor arrangement 400 in accordance with a preferred exemplary embodiment of the invention.

In accordance with the biosensor arrangement 400, macromolecular biomolecules can be detected using a reduction-oxidation recycling method, a reference electrode according to the invention keeping an analyte at a constant and defined electrical potential.

FIG. 4A shows the biosensor arrangement 400 having a first working electrode 401 and a second working electrode 402, which are integrated in a silicon substrate 403. A holding region 404 made of gold material is applied on the first working electrode 401. The holding region 404 serves for immobilizing DNA probe molecules 405 on the first working electrode 401. Such a holding region is not provided on the second working electrode 402.

If the biosensor arrangement 400 is intended to be used to detect DNA strands 407 having a base sequence that is complementary to the sequence of the immobilized DNA probe molecules 405, then the biosensor arrangement 400 is brought into contact with a solution to be examined, namely an electrolyte 406, in such a way that DNA strands 407 that have a sequence complementary to the sequence of the DNA probe molecules 405 and are possibly contained in the solution 406 to be examined can hybridize.

In order to be able to operate the biosensor arrangement 400 under defined conditions, in particular in order to keep the electrical potential of the electrolyte 406 constant, an integrated reference electrode arrangement 415 is integrated in the silicon substrate 403. The integrated reference electrode arrangement 415 has a circuit 416 integrated in the silicon substrate 403. Furthermore, the integrated reference electrode arrangement 415 has a reference electrode formed on the silicon substrate 403 and comprising a silver core 418 that is partly surrounded by a silver chloride sheathing 419 made of sparingly soluble silver chloride salt. The integrated circuit 416 is electrically coupled to the silver core 418 via an electrically conductive tungsten coupling means 417. The integrated circuit 416 is set up in such a way that a signal which is characteristic of the electrical potential of the electrolyte 406 is provided to said integrated circuit by the reference electrode. Furthermore, the circuit 416 is coupled, via another tungsten coupling means 420 to a counterelectrode 421 arranged on the silicon substrate 403. The circuit 416 is furthermore set up in such a way that it provides a control signal on the basis of the detected potential to the counterelectrode 421 immersed in the electrolyte 406. Said control signal is chosen in such a way that the electrical potential of the electrolyte 406 is influenced by means of the counterelectrode 421 in such a way that the electrical potential of the electrolyte 406 is equal to a reference value.

FIG. 4B shows a scenario in accordance with which the solution 406 to be examined contains DNA strands 407 to be detected, one of which has hybridized with a DNA probe molecule 405. The DNA strands 407 in the solution to be examined are marked with an enzyme 408, which makes it possible to cleave molecules described below into electrochemically activated partial molecules. The number of DNA probe molecules 405 provided is usually considerably greater than the number of DNA strands 407 to be determined that is contained in the solution 406 to be examined.

Once the DNA strands 407 contained in the solution 406 to be examined together with the enzyme 408 have hybridized with the immobilized DNA probe molecules 405, the biosensor arrangement 400 is preferably specially rinsed, the rinsing solution used for this being set up in such a way that such DNA strands at which a hybridization event has not taken place are removed, and the biosensor arrangement 400 is thereby cleaned of the solution 406 to be examined. The rinsing solution used for rinsing is admixed with an electrochemically inactive substance containing molecules which can be cleaved by means of the enzyme 408 into two partial molecules 410, at least one of which is electrochemically active and usually has an electrical charge.

As shown in FIG. 4C, the partial molecules 410 that are negatively charged in accordance with the exemplary embodiment described are attracted to the positively charged first working electrode 201, which is indicated by means of an arrow 411 in FIG. 4C. The negatively charged partial molecules 410 are oxidized at the first working electrode 401, which has a positive electrical potential, and are attracted as oxidized partial molecules 413 to the negatively charged second working electrode 402, where they are reduced again. The reduced partial molecules 414 migrate again to the positively charged working electrode 401. In this way, an electric circulating current is generated which is proportional to the number of charge carriers respectively generated by means of the enzymes 408.

The oxidation or reduction potentials are intended to be monitored during the operation of the biosensor arrangement 400. This requires application of a known electrical potential. This is clearly effected by means of the potentiostat device formed by the reference electrode, the counterelectrode 421 (and the working electrodes 401, 402).

As emerges from the above description, the functionality of the reference electrode arrangement 415 according to the invention is essential to the functionality of the biosensor arrangement 400.

The following publications are cited in this document:
[1] Homepage of the company Inventus Biotec, http://inventus-biotec.com/deu/produkt/t-sensorchip.html (as at Apr. 9, 2001)
[2] Homepage of the company World Precision Instruments, “Dri-Ref™ Referenz-Elektroden” [“Dri-Ref™ reference electrodes”], http://www.wpiinc.com/WPI Web/Biosensing/Dri-Ref.html (as at Apr. 9, 2001)
[3] Mace, M L, Montagu, J, Rose, S D, McGuinness, G “Novel Microarray Printing and Detection Technology” in: Schena, M (ed.) “Microarray Biochip Technology” (chapter 3), Biotechnique Books, Eaton Publishing, ISBN 1881299376
[4] http://pubs.acs.org/subscribe/journals/mdd/v04/i05/html/o5zubritskytablel.html (as at Apr. 9, 2001)
[5] DE 196 21 996 C2
[6] DE 196 21 997 C1
[7] U.S. Pat. No. 5,672,257
[8] EP 0 588 153 B1
[9] U.S. Pat. No. 5,801,428

### LIST OF REFERENCE SYMBOLS

100 Electrochemical experimental arrangement

101 Container

102 Analyte

103 First working electrode

104 Second working electrode

105 Current supply

106 Ammeter

107 Volt meter

108 Capillary

109 Silver/silver chloride electrode arrangement

110 Silver wire

200 Sensor arrangement

201 Working electrode

202 Counterelectrode

203 Reference electrode

204 Contact-connection

300 Layer sequence

301 Silicon substrate

302 Integrated circuit

303 Tungsten contact-connection element

304 Gold basic electrode

310 Layer sequence

311 Silver material

320 Circuit arrangement

321 Silver chloride sheathing

400 Biosensor arrangement

401 First working electrode

402 Second working electrode

403 Silicon substrate

404 Holding region

405 DNA probe molecule

406 Electrolyte

407 DNA strand

408 Enzyme

409 Cleavable molecule

410 Partial molecules

411 Arrow

412 Further solution

413 Oxidized first partial molecule

414 Reduced first partial molecule

415 Integrated reference electrode arrangement

416 Circuit

417 Tungsten coupling means

418 Silver core

419 Silver chloride sheathing

420 Other tungsten coupling means

421 Counterelectrode

1. (canceled)

2-12. (canceled)

13. A method for producing a biosensor circuit comprising:
forming an integrated circuit in a substrate;
forming a core of an integrated reference electrode by means of printing silver material as metal on the substrate;
applying biological molecules by means of printing on sensor arrays of the biosensor circuit arrangement, whereby the sensor arrays are biologically activated;
effecting the printing of silver material on the substrate and the printing of the biological molecules on the sensor arrays are effected in the same work step;
subsequently, at least partially surrounding the core made of silver material by a sheath made of a sparingly soluble salt of the silver material, thereby forming the integrated reference electrode; and
electrically coupling the integrated circuit to the core of the integrated reference electrode.

14. The method of claim 13, further comprising forming an electrically conductive coupling structure in such a way that the integrated circuit is electrically coupled to the core by means of said coupling structure.

15. The method of claim 14, further comprising arranging the coupling structure adjacent the substrate in such a way that the core is formed by means of covering the coupling structure with the silver material.

16. The method of claim 13, further comprising using silver chloride as the salt of the metal.

17. The method of claim 13, further comprising at least partially surrounding the core by the sheath by chlorinating the core made of silver using one of an electrochemical method and a chemical method.

18. The method of claim 13, further comprising configuring the circuit such that a signal characteristic of the electrical potential in a region surrounding the reference electrode can be provided to said circuit by the reference electrode.

19. The method of claim 13, wherein the substrate used is one a group comprising a semiconductor material, glass, plastic, and ceramic.

20. The method of claim 14, wherein the coupling structure is formed from one of gold and platinum.

21. A method for producing a biosensor circuit comprising:
forming an integrated circuit is formed in a substrate;
forming a core of an integrated reference electrode made of silver as metal by printing silver salt material on the substrate and chemically reducing the silver salt material to form silver;
applying biological molecules by means of printing on sensor arrays of the biosensor circuit arrangement, whereby the sensor arrays are biologically activated;
effecting the printing of the core of the integrated reference electrode on the substrate and the printing of the biological molecules on the sensor arrays in the same work step;
subsequently, at least partially surrounding the core of the integrated reference electrode by a sheath made of a sparingly soluble salt of the silver as metal, thereby forming the integrated reference electrode; and
electrically coupling the integrated circuit to the core of the integrated reference electrode.

22. The method of claim 21, further comprising forming an electrically conductive coupling structure in such a way that the integrated circuit is electrically coupled to the core by means of said coupling structure.

23. The method of claim 22, further comprising arranging the coupling structure adjacent the substrate in such a way that the core is formed by means of covering the coupling structure with the silver material.

24. The method of claim 21, further comprising using silver chloride as the salt of the metal.

25. The method of claim 21, further comprising at least partially surrounding the core by the sheath by chlorinating the core made of silver using one of an electrochemical method and a chemical method.

26. The method of claim 21, further comprising configuring the circuit such that a signal characteristic of the electrical potential in a region surrounding the reference electrode can be provided to said circuit by the reference electrode.

27. The method of claim 21, wherein the substrate used is one a group comprising a semiconductor material, glass, plastic, and ceramic.

28. The method of claim 22, wherein the coupling structure is formed from one of gold and platinum.

29. A biosensor circuit comprising:
an integrated circuit in a substrate;
an integrated reference electrode in the integrated circuit formed on the substrate;
a core of the integrated reference electrode that is made of silver metal and is at least partially surrounded by a sheath made of a sparingly soluble salt of the silver metal; and
sensor arrays including biological molecules;
wherein the integrated circuit is electrically coupled to the core of the integrated reference electrode.

30. The biosensor circuit of claim 29, wherein the core of the integrated reference electrode is formed by printing silver material as metal on the substrate.

31. The biosensor circuit of claim 30, wherein the biological molecules are applied by printing the sensor arrays.

32. The biosensor circuit of claim 31, wherein the printing of the silver material and the biological molecules occurs in on work step.
